(19) United States
(12) Patent Application Publication
(10) Pub. No.: US 2025/0205394 A1
(43) Pub. Date: Jun. 26, 2025

(54) SEPARATING MEMBRANE FOR DENTISTRY AND METHOD FOR MANUFACTURING THE SAME

(71) Applicant: NAN YA PLASTICS CORPORATION, TAIPEI (TW)

(72) Inventors: TE-CHAO LIAO, TAIPEI (TW); Min-Fan Chung, Taoyuan City (TW); CHING-YAO YUAN, TAIPEI (TW); YI-CHEN TSAI, TAIPEI (TW)

(21) Appl. No.: 18/599,375

(22) Filed: Mar. 8, 2024

(30) Foreign Application Priority Data
Dec. 22, 2023 (TW) 112150186

(51) Int. Cl. A61L 27/56; A61L 27/12; A61L 27/36; A61L 27/52

(52) U.S. Cl. CPC A61L 27/56; A61L 27/12; A61L 27/3608; A61L 27/52

(57) ABSTRACT

A separating membrane for dentistry and a method for manufacturing the same are provided. The separating membrane for dentistry includes a porous layer and a hydrophilic layer encapsulating the porous layer. The porous layer includes a porous structure and a bone regeneration material attached on the porous structure. Based on a total weight of the porous layer being 100 wt %, a weight ratio of the porous structure ranges from 22 wt % to 50 wt %, and a weight ratio of the bone regeneration material ranges from 50 wt % to 78 wt %. The porous structure is formed from a biodegradable polymer. The hydrophilic layer is formed from a hydrophilic material. The separating membrane is used to contact an alveolar bone or bone grafts.

### CROSS-REFERENCE TO RELATED PATENT APPLICATION

This application claims the benefit of priority to Taiwan Patent Application No. 112150186, filed on Dec. 22, 2023. The entire content of the above identified application is incorporated herein by reference.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The present disclosure relates to a separating membrane for dentistry and a method for manufacturing the same, and more particularly to a separating membrane for dentistry and a method for manufacturing the same which can be used on an alveolar bone.

### BACKGROUND OF THE DISCLOSURE

A guided bone regeneration (GBR) procedure, which is also called a bone repair operation, is usually performed before a dental implant. This can solve problems caused by having a tooth missing for an extended period of time, such as shrinkage of an alveolar bone.

Referring to FIG. 4, during the GBR, a gum tissue G is cut apart, and then bone grafts B are filled in a depression of the alveolar bone, so as to facilitate hyperplasia of bone cells in a tooth ridge R. In a case where a growing space for the bone cells is occupied by the gum tissue G or soft tissues during cell proliferation, a separating membrane F is disposed on the bone grafts B to separate the alveolar bone from the soft tissues. Finally, the gum tissue G is stitched up. Accordingly, the bone cells can grow within a specific space for rebuilding the tooth ridge R.

A common separating membrane that is currently available on the market is made from collagen (hereinafter referred to as a collagen membrane). Since physical properties of the collagen membrane are weak, the collagen membrane is likely to rupture after the implant, which causes the artificial bones to fall. Moreover, the collagen membrane is not moldable. In order to completely cover a wound, dentists need to fix the shape of the collagen membrane by sewing or other auxiliary measures. Accordingly, the collagen membrane is inconvenient for use due to its weak physical properties.

Therefore, how to improve the physical properties of the conventional separating membrane and increase its convenience of use, so as to overcome the above-mentioned problems, has become one of the important issues to be addressed in the industry.

### SUMMARY OF THE DISCLOSURE

In response to the above-referenced technical inadequacy, the present disclosure provides a separating membrane for dentistry and a method for manufacturing the same.

In order to solve the above-mentioned problems, one of the technical aspects adopted by the present disclosure is to provide a separating membrane for dentistry. The separating membrane for dentistry includes a porous layer and a hydrophilic layer encapsulating the porous layer. The porous layer includes a porous structure and a bone regeneration material attached on the porous structure. Based on a total weight of the porous layer being 100 wt %, a weight ratio of the porous structure ranges from 22 wt % to 50 wt %, and a weight ratio of the bone regeneration material ranges from 50 wt % to 78 wt %. The porous structure is formed from a biodegradable polymer. The hydrophilic layer is formed from a hydrophilic material. The separating membrane is used to contact an alveolar bone or bone grafts.

In one of the possible or preferred embodiment, a thickness of the separating membrane ranges from 100 μm to 300 μm.

In one of the possible or preferred embodiment, a porosity of the porous layer ranges from 10% to 30%.

In one of the possible or preferred embodiment, the bone regeneration material includes calcium phosphate.

In one of the possible or preferred embodiment, a particle size of the bone regeneration material ranges from 10 μm to 30 μm.

In one of the possible or preferred embodiment, the biodegradable polymer includes polylactic acid, and an amount of polylactic acid in the biodegradable polymer is higher than or equal to 50 wt %.

In one of the possible or preferred embodiment, a weight average molecular weight of the biodegradable polymer ranges from 100,000 g/mol to 600,000 g/mol.

In one of the possible or preferred embodiment, the hydrophilic material includes hyaluronic acid.

In another aspect, the present disclosure provides a method for manufacturing a separating membrane. The method includes: preparing a polymer solution including a solvent, a biodegradable polymer, and a bone generation material; using the polymer solution for an electrospinning process to form a porous layer; and immersing the porous layer into a treatment solution, so as to form a hydrophilic layer encapsulating the porous layer. Based on a solid content of the polymer solution being 100 wt %, an amount of the biodegradable polymer ranges from 22 wt % to 50 wt %, and an amount of the bone generation material ranges from 50 wt % to 78 wt %.

In one of the possible or preferred embodiment, the solvent is selected from the group consisting of: acetone, methyl ketone, ethylene glycol, isopropyl alcohol, deacetylated chitosan, N,N-dimethylformamide, dimethylacetamide, dimethyl sulfoxide, and ethyl ether.

In one of the possible or preferred embodiment, in the electrospinning process, an ejection speed of the polymer solution ranges from 5 ml/hr to 10 ml/hr.

In one of the possible or preferred embodiment, the treatment solution contains 10 wt % to 30 wt % hyaluronic acid.

Therefore, in the separating membrane for dentistry and the method for manufacturing the same provided by the present disclosure, by virtue of “based on the total weight of the porous layer being 100 wt %, a weight ratio of the porous structure ranging from 22 wt % to 50 wt %, and a weight ratio of the bone regeneration material ranging from 50 wt % to 78 wt %,” the separating membrane for dentistry can help the growth of the bone cells.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The described embodiments may be better understood by reference to the following description and the accompanying drawings, in which:

FIG. 1 is a schematic cross-sectional side view of a separating membrane for dentistry according to the present disclosure;

FIG. 2 is a scanning electron microscope image of the separating membrane for dentistry according to the present disclosure;

FIG. 3 is a flowchart of a method for manufacturing the separating membrane for dentistry according to the present disclosure; and

FIG. 4 is a schematic view of guided bone regeneration according to the present disclosure.

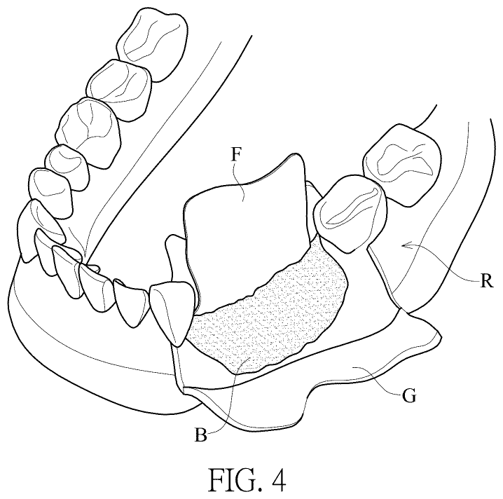

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a”, “an”, and “the” includes plural reference, and the meaning of “in” includes “in” and “on”. Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first”, “second” or “third” can be used to describe various components, signals or the like, which are for distinguishing one component/signal from another one only, and are not intended to, nor should be construed to impose any substantive limitations on the components, signals or the like.

The separating membrane for dentistry of the present disclosure can be used as a separating membrane between bone grafts and soft tissues in guided bone regeneration. Since the separating membrane for dentistry has good physical properties and flexibility, the problem of rupture of the separating membrane during a dental implant can be improved. In addition, the separating membrane for dentistry can be bent and disposed along with a shape of an alveolar bone and bone grafts. The separating membrane for dentistry of the present disclosure contains a bone regeneration material, such that the separating membrane can also be used to replace a part of the bone grafts besides being a separating membrane.

### First Embodiment

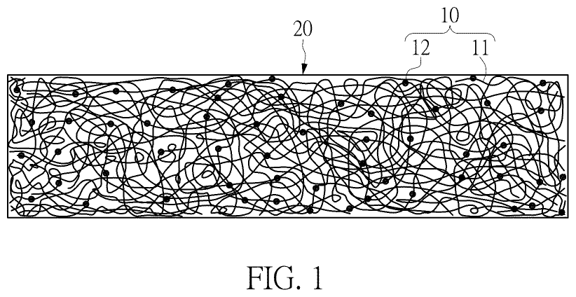

Referring to FIG. 1, the separating membrane for dentistry of the present disclosure includes a porous layer 10 and a hydrophilic layer 20. The porous layer 10 is encapsulated by the hydrophilic layer 20.

The porous layer 10 is a main structure of the separating membrane for dentistry. The porous layer 10 can separate epithelial cells from bone cells, so as to prevent the proliferated epithelial cells from occupying a growth space for the bone cells. The porous layer 10 has a good physical properties and flexibility, thereby enhancing physical properties of the overall separating membrane for dentistry.

In consideration of a usage convenience and a mechanical strength of the separating membrane for dentistry, a thickness of the separating membrane for dentistry can range from 100 μm to 300 μm, such as 125 μm, 150 μm, 175 μm, 200 μm, 225 μm, 250 μm, or 275 μm.

The porous layer 10 includes a porous structure 11 and a bone regeneration material 12. The bone regeneration material 12 is attached on the porous structure 11.

The porous structure 11 is formed from a plurality of polymer fibers winded up or stacked up with each other. In a winding or stacking process, the plurality of polymer fibers cannot be regularly and uniformly winded or stacked. In this way, holes are formed between the plurality of polymer fibers and forms the porous structure 11.

The holes of the porous structure 11 provide channels for blood or nutrients to pass by; while, a quantity of the holes cannot be too large or too small. Higher porosity of the porous structure 11 decreases a structural strength of the separating membrane, which is inconvenient for doctor to use in surgery. In addition, soft tissues, such as gum tissues and oral mucosa tissues may penetrate the separating membrane for dentistry and occupy the growth space for the bone cells. On the other hand, lower porosity of the porous structure 11 is unfavorable to the passage for blood or nutrients.

Specifically, the porosity of the porous layer 10 can range from 10% to 30%, and preferably range from 15% to 25%. For example, the porosity of the porous layer 10 can be 12.5%, 15%, 17.5%, 20%, 22.5%, 25%, or 27.5%.

The porous structure 11 is formed from a biodegradable polymer. For example, the biodegradable polymer can be selected from the group consisting of: polylactic acid (PLA), polycaprolactone (PCL), polyhydroxyalkanoate (PHA), and polyglycolic acid (PGA). From a perspective of biocompatibility, the biodegradable polymer is preferably polylactic acid. In an exemplary embodiment, the biodegradable polymer is a mixture of polylactic acid and other biodegradable polymers.

The separating membrane for dentistry is supported by the porous structure 11. Hence, the biodegradable polymer should have sufficient mechanical strength. In an exemplary embodiment, a molecular weight of the biodegradable polymer can range from 100,000 g/mol to 600,000 g/mol, and preferably from 150,000 g/mol to 350,000 g/mol. For example, the molecular weight of the biodegradable polymer can be 200,000 g/mol, 250,000 g/mol, or 300,000 g/mol.

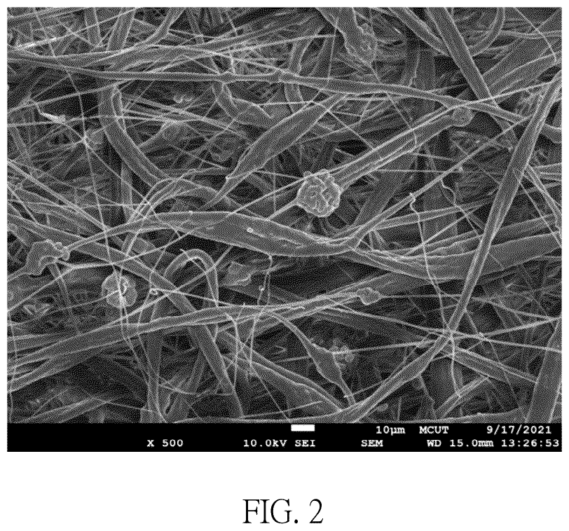

The bone regeneration material 12 can help the growth of the bone cells, and can also replace a part of the bone grafts. Therefore, the separating membrane for dentistry of the present disclosure upholds both effects of a separating membrane and bone grafts. Specifically, the bone regeneration material 12 can be attached on the porous structure 11, and also be fixed in the holes of the porous structure 11. A scanning electron microscope image of the separating membrane for dentistry is shown in FIG. 2.

When the separating membrane for dentistry is used, the bone regeneration material 12 is slowly released from the separating membrane for dentistry to promote the growth of the bone cells. Therefore, a relationship between the porosity of the porous structure 11 and the size of the bone regeneration material 12 is important. A carrying amount of the bone regeneration material 12 and a releasing rate of the bone regeneration material 12 are both influenced by the porosity of the porous structure 11 and the size of the bone regeneration material 12.

In an exemplary embodiment, the bone regeneration material 12 includes calcium phosphate (Ca₃(PO₄)₂). Specifically, the bone regeneration material 12 is an osteoinductive calcium phosphate (β-tricalcium phosphate, β-TCP), which can accelerate the growth of the bone cells.

The bone regeneration material 12 can exist in a form of particles. A size of the bone regeneration material 12 can range from 10 μm to 30 μm. In this size range, the bone regeneration material 12 can be fixed on the porous structure 11, thereby facilitating the growth of the bone cells. When the size of the bone regeneration material 12 is too large, the bone regeneration material 12 has difficulty to be facilitated by the bone cells. When the size of the bone regeneration material 12 is too small, the bone regeneration material 12 has difficulty to be carried on the porous structure 11. Therefore, the size of the bone regeneration material 12 can be 12 μm, 14 μm, 16 μm, 18 μm, 20 μm, 22 μm, 24 μm, 26 μm, or 28 μm.

In the present disclosure, an amount of the bone regeneration material 12 in the porous layer 10 is higher than 50 wt %. The high amount of the bone regeneration material 12 enables the separating membrane for dentistry to replace a part of the bone grafts. In addition, the addition of the bone regeneration material 12 can also enhance the mechanical strength of the separating membrane for dentistry.

Specifically, based on the total weight of the porous layer 10 being 100 wt %, the amount of the porous structure 11 ranges from 22 wt % to 50 wt %, and the amount of the bone regeneration material 12 ranges from 50 wt % to 78 wt %.

It should be noted that the amount of the bone regeneration material 12 is not the higher the better. When the bone regeneration material 12 is excessive, the growth of bone cells is not as good as expected. The experimental data of the growth of bone cells under conditions of different amounts of the bone regeneration material 12 are provided later.

In other embodiments, the amount of the porous structure 11 can be 25 wt %, 30 wt %, 35 wt %, 40 wt %, or 45 wt %, and the amount of the bone regeneration material 12 can be 55 wt %, 60 wt %, 65 wt %, 70 wt %, or 75 wt %.

The porous structure 11 is encapsulated by the hydrophilic layer 20. Specifically, the hydrophilic layer 20 is attached on surfaces of the porous structure 11 and the bone regeneration material 12, so as to enhance hydrophilicity of the separating membrane for dentistry. The disposition of the hydrophilic layer 20 enhances the usage convenience of the separating membrane for dentistry. The separating membrane for dentistry can be easily attached onto the alveolar bone or the bone grafts, and blood and nutrients can easily pass through the separating membrane for dentistry.

Besides completely encapsulating the porous layer 10, a part of the hydrophilic layer 20 can also be disposed in the porous layer 10 to fill the holes of the porous structure 11, thereby facilitating the passage of hydrophilic materials and nutrients.

The hydrophilic layer 20 is formed from a hydrophilic material, preferably being substances that are not rejected by the human body. The hydrophilic material can be selected from the group consisting of hyaluronic acid and its derivatives, and water-soluble vitamins, such as vitamin C and vitamin B complex. Preferably, the hydrophilic material is hyaluronic acid. By the disposition of the hydrophilic layer 20, the separating membrane for dentistry of the present disclosure can be quickly turned into a moldable and softened state by absorbing water through the hydrophilic layer 20.

A water contact angle of an outer surface of the separating membrane for dentistry is less than 80°, which greatly shortens the soaking and wetting time before using and enhances the moldability and encapsulating ability after being fully wetted. In the present disclosure, the contact angle of the outer surface of the separating membrane for dentistry is preferably less than 60°, more preferably less than 30°, and most preferably less than 10°.

However, without seriously conflicting with the effects of the present disclosure, other hydrophilic materials, such as hydroxyl-containing substances, carboxylic acid group-containing substances, sulfonic acid group-containing substances, ether group-containing substances, epoxy group-containing substances, and amine group-containing substances can also be used to perform a hydrophilization treatment.

Supplementary description, when the hydrophilic material is hyaluronic acid, the molecular weight of the hyaluronic acid is not limited, but preferably ranges from 10,000 g/mol to 1,000,000 g/mol, and more preferably ranges from 10,000 g/mol to 700,000 g/mol. When the molecular weight of the hyaluronic acid is lower than 10,000 g/mol, the hydrophilic layer 20 has difficulty to be attached on the porous layer 10. When the molecular weight of the hyaluronic acid is higher than 1,000,000 g/mol, the hyaluronic acid has difficulty to permeate into the porous layer 10.

The hydrophilic layer 20 encapsulating the porous layer 10 is very thin, such that a thickness of the separating membrane for dentistry ranges from 100 μm to 300 μm, which is almost equal to the thickness of the porous layer 10. For example, the thickness of the separating membrane for dentistry can be 125 μm, 150 μm, 175 μm, 200 μm, 225 μm, 250 μm, or 275 μm.

A stretching stress of the separating membrane for dentistry of the present disclosure measured at a temperature of 25° C. and a relative humidity of 50% ranges from 0.3 MPa to 5 MPa. An adhesion strength of the separating membrane for dentistry of the present disclosure measured by the ASTM D3121-2006 standard ranges from 0.3 N to 0.7 N.

It should be noted that although the guided bone regeneration is used as an example to describe the advantages of the separating membrane for dentistry of the present disclosure, the separating membrane for dentistry of the present disclosure can also be used in other surgeries that are related to the human body.

### Second Embodiment

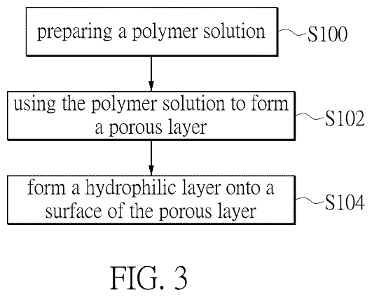

Referring to FIG. 3, the separating membrane for dentistry according to the first embodiment can be manufactured by a method for manufacturing the separating membrane for dentistry according to a second embodiment. The method includes steps: preparing a polymeric solution (step S100), forming a porous layer by using the polymeric solution (step S102), and forming a hydrophilic layer onto a surface of the porous layer (step S104). Detailed descriptions of each step are provided below with reference of FIG. 1.

In step S100, the polymeric solution includes the bone regeneration material, the biodegradable polymer, and a solvent.

A solid content of the polymeric solution can be adjusted according to various manners in step S102. Specifically, the porous layer can be formed through a non-woven spinning technology, a freeze-drying technology, or an electrospinning technology. In the second embodiment, the porous layer formed through the electrospinning technology is taken as an example. In order to adapt the manner of electrospinning, based on the total weight of the polymeric solution being 100 wt %, the solid content of the polymeric solution ranges from 1 wt % to 50 wt %, and an amount of the solvent ranges from 50 wt % to 99 wt %.

Solid components of the polymeric solution include the biodegradable polymer and the bone regeneration material. In the present disclosure, based on a total solid content of the polymeric solution being 100 wt %, the amount of the biodegradable polymer ranges from 22 wt % to 50 wt %, and the amount of the bone regeneration material ranges from 50 wt % to 78 wt %.

In practical application, the solvent can be selected from the group consisting of: acetone, methyl ketone, ethylene glycol, isopropyl alcohol, deacetylated chitosan (DAC), N,N-dimethylformamide (DMF), dimethylacetamide (DMAC), dimethyl sulfoxide (DMSO), and ethyl ether. Preferably, the solvent can be acetone or a mixture of acetone and dimethylacetamide.

Calcium phosphate composite particles and polylactic acid particles can respectively be used as materials of the biodegradable polymer and the bone regeneration material. Specifically, the calcium phosphate composite particles include calcium phosphate and polylactic acid, and the polylactic acid particles include 100 wt % polylactic acid. Therefore, the solid components of the polymeric solution include calcium phosphate and polylactic acid.

Specifically, based on a total weight of the calcium phosphate composite particles being 100 wt %, an amount of the calcium phosphate ranges from 10 wt % to 90 wt %, but the present disclosure is not limited thereto. In order to control the weight ratio of the bone regeneration material to the biodegradable polymer, a weight ratio of the polylactic acid particles to the calcium phosphate composite particles ranges from 1:0.9 to 1:5.

In step S102, the porous layer is formed from the polymeric solution through electrospinning technology. After being electrospun and dried, the porous layer 10 is formed from the solid components of the polymeric solution. The porous structure 11 is formed from polylactic acid, and calcium phosphate is attached on the porous structure 11 or filled in the holes of the porous structure 11.

In an electrospinning process, the prepared polymeric solution is added into a storage tank, and a nozzle and a collection plate are electrically connected with a positive electrode and a negative electrode of a high-voltage power supply respectively. After applying a high-voltage power, the polymeric solution is sprayed from the nozzle. In an electrostatic field, the polymeric solution is solidified to form polymer fibers and deposited on the collection plate. By controlling the movement of the nozzle, the polymer fibers can be tightly stacked, entangled, or intertwined along a specific direction, thereby forming a porous layer 10 with a uniform thickness.

Subsequently, a drying process is implemented. The solvent in the polymeric solution is evaporated, leaving the porous structure 11 and the bone regeneration material 12 to form the porous layer 10.

Parameters used in the electrospinning process include: an electrospinning temperature ranging from 5° C. to 95° C. and preferably ranging from 10° C. to 90° C.; a voltage of the high-voltage power ranges from 10 kV to 36 kV; an ejection rate of the polymeric solution ranging from 5 cc/hr to 10 cc/hr; and a collection distance between the nozzle and the collection plate ranging from 15 cm to 90 cm.

In step S104, the porous layer 10 is immersed in a treatment solution at room temperature. The treatment solution contains a hydrophilic material. After being immersed, the hydrophilic material is attached on the surface of the porous layer 10 and permeated into the porous layer 10, thus the porous layer 10 is encapsulated by the hydrophilic layer 20.

Subsequently, another drying process is implemented so as to evaporate the solvent of the treatment solution, leaving the hydrophilic material attached on the porous structure 11 and the bone regeneration material 12 which forms the hydrophilic layer 20.

Based on a total weight of the treatment solution being 100 wt %, an amount of the hydrophilic material can range from 10 wt % to 30 wt %. Preferably, the amount of the hydrophilic material can be 15 wt %, 20 wt %, 25 wt %, or 30 wt %. In an exemplary embodiment, the hydrophilic material is hydraulic acid. The treatment solution contains 95% ethanol, water and hydraulic acid, in which a weight ratio of 95% ethanol to water ranges from 1:1 to 3:2.

### EXPERIMENTAL DATA

In order to prove that the separating membrane for dentistry of the present disclosure can help the growth of the bone cells, the separating membranes for dentistry containing different amounts of the bone regeneration material (i.e., calcium phosphate) are manufactured. An osteocyte differentiation test is implemented on the separating membranes for dentistry, and the results are listed in Table 1 and Table 2. In addition, a tensile strength test is implemented on the separating membranes for dentistry, and the results are listed in Table 3.

In Table 1, Table 2, and Table 3, the difference between Examples and Comparative Examples is the content of the bone regeneration material.

In the osteocyte differentiation test shown in Table 1 and Table 2, the separating membrane for dentistry is cut into square pieces with a side length of 1 cm. The square pieces are disposed in a 24-well plate. 60λ of bone marrow mesenchymal stem cell (BMSC) containing 3×10⁴ cells are planted on the square pieces, and the cells are cultured in an environment of 37° C. and a carbon dioxide concentration of 5% for one hour, so as to adhere the cells onto the square pieces.

When the cells have adhered to the square pieces with confirmation, 500λ of culture medium is added into the 24-well plate, and placed in an environment of 37° C. and a carbon dioxide concentration of 5% overnight. Subsequently, the culture medium in the 24-well plate is removed and replaced by an osteoinductive culture medium, and then cultured in an environment of 37° C. and a carbon dioxide concentration of 5%. The replacement of the fresh osteoinductive culture medium is carried out each two or three days. A total culture time is 12 days or 14 days.

After 12 days or 14 days of culture, the medium in the 24-well plate is removed, and the cells are washed by phosphate buffered saline (PBS). 500λ of 5 mg/ml para-nitrophenylphosphate (p-NPP) is added into the 24-well plate, and the 24-well plate is placed in dark at room temperature for 45 minutes. 100λ of p-NPP is sampled, and an absorbance value at a wavelength of 405 nm of p-NPP is measured.

In Table 3, the tensile strength (dry) indicates that the separating membrane for dentistry is measured at a temperature of 25° C. and an absolute humidity of 50%. The tensile strength (wet) indicates that the separating membrane for dentistry is measured after immersed in 37° C. normal saline for 30 minutes.

TABLE 1

| | Comparative | | |
|---|---|---|---|
| | Example 1 | Example 1 | Example 2 |
| Content of bone generation | 0 | 48 | 75 |
| material in porous layer (wt %) | | | |
| Absorbance value after 14 | 1.0 | 1.5 | 1.75 |
| days culture (−) | | | |

TABLE 2

| | Comparative | | |
|---|---|---|---|
| | Example 2 | Example 3 | Example 4 |
| Content of bone generation | 0 | 75 | 80 |
| material in porous layer (wt %) | | | |
| Absorbance value after 12 | 1.8 | 1.9 | 1.2 |
| days culture (−) | | | |

TABLE 3

| | Example | Example | Example | Comparative |
|---|---|---|---|---|
| | 5 | 6 | 7 | Example 3 |
| Content of bone generation | 50 | 66.6 | 75 | 0 |
| material in porous layer (wt %) | | | | |
| Thickness of separating | 150 | 150 | 100 | 350 |
| membrane for dentistry (μm) | | | | |
| Tensile strength (dry) | 7.9 | 7.8 | 8.6 | 5.3 |
| (Kgf/cm²) | | | | |
| Tensile strength (wet) | 4 | 3.5 | 4.2 | 7.4 |
| (Kgf/cm²) | | | | |

According to the results in Table 1, the bone generation material in the separating membrane for dentistry can help the growth of the bone cells. In addition, the higher the concentration of the bone generation material is, the better the promotion of the growth of the bone cells is.

However, according to the results in Table 2, when the concentration of the bone generation material is too high, the growth of the bone cells will be inhibited instead. Therefore, the preferable amount of the bone generation material in the porous layer ranges from 50 wt % to 78 wt %.

According to the results in Table 3, besides the promotion of the growth of the bone cells, the addition of the bone generation material can also enhance the mechanical strength of the separating membrane for dentistry. Even if the thickness of the separating membrane for dentistry is thin, the separating membrane for dentistry can still have a good tensile strength at a dry state.

### Beneficial Effects of the Embodiments

In conclusion, in the separating membrane for dentistry and the method for manufacturing the same provided by the present disclosure, by virtue of “based on the total weight of the porous layer being 100 wt %, a weight ratio of the porous structure ranging from 22 wt % to 50 wt %, and a weight ratio of the bone regeneration material ranging from 50 wt % to 78 wt %,” the separating membrane for dentistry can help the growth of the bone cells.

Further, the separating membrane for dentistry of the present disclosure contains a high content of the bone generation material. The bone generation material can help the growth of the bone cells and replace a part of the bone grafts. Therefore, the separating membrane for dentistry can be directly disposed on a depression of an alveolar bone or the bone grafts.

Moreover, the disposition of the hydrophilic layer enables the separating membrane for dentistry of the present disclosure to turn into a moldable and softened state by absorbing water in a short period of time (5 minutes), so as to adapt to different three-dimensional shapes. In addition, the separating membrane for dentistry of the present disclosure can be firmly attached on an affected area (such as a shrinkage of the alveolar bone) to provide sufficient growth space for repair, regeneration, and integration.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A separating membrane for dentistry, comprising:
a porous layer including a porous structure and a bone regeneration material attached on the porous structure; wherein the porous structure is formed from a biodegradable polymer; wherein, based on a total weight of the porous layer being 100 wt %, a weight ratio of the porous structure ranges from 22 wt % to 50 wt %, and a weight ratio of the bone regeneration material ranges from 50 wt % to 78 wt %; and
a hydrophilic layer encapsulating the porous layer; wherein the hydrophilic layer is formed from a hydrophilic material;
wherein the separating membrane is used to contact an alveolar bone or bone grafts.

2. The separating membrane according to claim 1, wherein a thickness of the separating membrane ranges from 100 μm to 300 μm.

3. The separating membrane according to claim 1, wherein a porosity of the porous layer ranges from 10% to 30%.

4. The separating membrane according to claim 1, wherein the bone regeneration material includes calcium phosphate.

5. The separating membrane according to claim 1, wherein a particle size of the bone regeneration material ranges from 10 μm to 30 μm.

6. The separating membrane according to claim 1, wherein the biodegradable polymer includes polylactic acid, and an amount of polylactic acid in the biodegradable polymer is higher than or equal to 50 wt %.

7. The separating membrane according to claim 1, wherein a weight average molecular weight of the biodegradable polymer ranges from 100,000 g/mol to 600,000 g/mol.

8. The separating membrane according to claim 1, wherein the hydrophilic material includes hyaluronic acid.

9. A method for manufacturing a separating membrane, comprising:
preparing a polymer solution including a solvent, a biodegradable polymer, and a bone generation material; wherein, based on a solid content of the polymer solution being 100 wt %, an amount of the biodegradable polymer ranges from 22 wt % to 50 wt %, and an amount of the bone generation material ranges from 50 wt % to 78 wt %;
using the polymer solution for an electrospinning process to form a porous layer; and
immersing the porous layer into a treatment solution, so as to form a hydrophilic layer encapsulating the porous layer.

10. The method according to claim 9, wherein the solvent is selected from the group consisting of: acetone, methyl ketone, ethylene glycol, isopropyl alcohol, deacetylated chitosan, N, N-dimethylformamide, dimethylacetamide, dimethyl sulfoxide, and ethyl ether.

11. The method according to claim 9, wherein, in the electrospinning process, an ejection speed of the polymer solution ranges from 5 ml/hr to 10 ml/hr.

12. The method according to claim 9, wherein the treatment solution contains 10 wt % to 30 wt % hyaluronic acid.
